SEQUENCE LISTING (2 sequences)

SEQ ID NO: 1 - protein, 15 aa, Camelus dromedarius
LKAMDPTPPLWIKTE

SEQ ID NO: 2 - protein, 10 aa, Camelus dromedarius
LKAMDPTPPL

(19) United States
(12) Patent Application Publication
(10) Pub. No.: US 2024/0285653 A1
(43) Pub. Date: Aug. 29, 2024

(54) COMPOSITIONS AND METHODS FOR TREATMENT AND PREVENTION OF DISEASES UTILIZING TIRILAZAD SALTS

(71) Applicant: Patrick T. PRENDERGAST, Byrock (AU)

(72) Inventor: Patrick T. PRENDERGAST, Byrock (AU)

(21) Appl. No.: 18/413,765

(22) Filed: Jan. 16, 2024

Related U.S. Application Data
(63) Continuation of application No. PCT/US2023/061743, filed on Feb. 1, 2023.

(30) Foreign Application Priority Data
Feb. 9, 2022 (IE) 2022/0049

(51) Int. Cl. A61K 31/58; A61K 45/06; A61P 43/00

(52) U.S. Cl. CPC A61K 31/58; A61K 45/06; A61P 43/00

(57) ABSTRACT

Provided is a method for treating or preventing laminitis comprising administering to an ungulate a therapeutically or prophylactically effective amount of a 21-aminosteroid, such as U-74389G/Methylated Tirilazad.

### CROSS-REFERENCE TO RELATED APPLICATIONS

This application is a bypass continuation of International Application No. PCT/US2023/061743, filed Feb. 1, 2023, which claims the benefit of priority of Irish Patent Application Serial No. 2022/0049, filed on Feb. 9, 2022, which are incorporated by reference herein in their entireties.

### INCORPORATION OF SEQUENCE LISTING

The sequence listing that is contained in the file named “BTL0002-201BC1-US,” which is 3,072 bytes as measured in Microsoft Windows operating system and was created on Jan. 30, 2023, is filed electronically herewith and incorporated herein by reference.

Laminitis (also termed founders) is inflammation of the laminae of the foot—the soft tissue that attaches the coffin or pedal bone of the foot to the hoof wall. Laminitis occurs especially in horses, but is also found in other ungulates, e.g., dairy cows. Since the second half of the 19^{th} century, several essays on laminitis have appeared in monographs, scientific papers, and textbooks. In the 19^{th} and 20^{th} centuries, scientific and technical progress increased available knowledge on the character and the pathogenesis of laminitis. New possibilities concerning diagnostics and therapy were established. However, up until today, laminitis remains one of the most important diseases, especially for horses, and even with new knowledge and therapies, a cure or preventive treatment of laminitis is not available today.

Laminitis is regarded as the most important claw disease of cattle. Laminitis, and the accompanying local trauma, has been made responsible for the development of sole ulcers. The immune system is vulnerable to inflammatory episodes; for example, the dairy cow is very prone especially around the time of birth (35 days before calving to 70 days after calving) because of massive changes in its metabolism caused by hormonal changes. Because of diagnostic uncertainties, and therefore limited possibilities to diagnose in practice, basic data for the frequency of the appearance of laminitis are missing in dairy cows during this period.

Concerning the pathogenesis of laminitis, the contacts of the lamellae become loose from the epidermis lamella, which physiologically grip into each other like a zipper. Local edema formation and swelling as a result of inflammatory signals from an activated immune system cause a higher outlet of tissue liquid and blood cells, results in high pressure between the coffin bone and the hoof wall. As there is no possibility of expansion, the high pressure causes heavy pain in that area for the animal. In this heavy pressure gradient, chronic laminitis results after only 48 hours. Further effects of the loosening of the contacts are the sinking and rotation of the coffin bone in the hoof capsule and a rotation of the coffin joint, up to a possible breakthrough of the tip of the coffin bone through the sole with hoof loss, which is a complete detachment of the hoof capsule.

The most common cause of laminitis is insulin resistance and a local inflammatory cascade from carbohydrate-rich food, but laminitis can also occur after birth, as a result of poisoning (e.g. through consuming poisonous mushrooms), too much exposure to very hard grounds, or medicaments (e.g. cortisone). There are also other clinical pictures that can cause laminitis, e.g., colic, enteritis, lumbago, thyroid diseases, and Cushing's syndrome. Also discussed as causes of laminitis are types of housing of the animals and their stress levels, allergies, toxins generated through too high of a protein supply, too much starch, or different kinds of sugars in high concentrations (e.g., fructans), consuming poisonous plants, pesticides, fertilizer, or overdoses of medicaments. More frequent factors that can induce laminitis include unnatural preserving agents and additives to nutrients that are not of the natural habitat of the animals in question. In relation to bovine, previous systemic diseases like rumen acid anemia and endometritis are known triggers for laminitis, which causes functional and morphological changes in the capsule due to the creation of an inflammatory cascade of protease enzymes and certain cytokines.

In addition to bovine, laminitis also occurs commonly in other ungulates from families like equine, deer, ovis, and capra.

Metalloproteinases and serine proteases are naturally occurring enzymes present in many tissues of the equine body and in mammals in general. These enzymes act to degrade proteins, normally in a controlled and specific manner. To prevent the uncontrolled destruction of target proteins and tissue, such as the hoof, the activity of these proteolytic enzymes is modulated by inhibitor serum peptides normally present under healthy conditions wherein the combined and balanced actions of proteases and their specific inhibitors act to control the level of biologically active and structurally important proteins of the body, thereby regulating many important physiological processes and maintaining the structural integrity of structures such as the hoof.

Metalloproteinases (MMPs) are an important group of proteinases. These enzymes are characterized by their requirement for the presence of a metal ion in order to catalyze proteolysis. Approximately 17 different metalloproteinases have been identified and/or cloned that share significant sequence homology. The metalloproteinase family can be subdivided into five groups according to their structural and functional properties: (i) the collagenases (metalloproteinases-1, 8, and 13); (ii) gelatinases A and B (metalloproteinase-2 and metalloproteinase-9); (iii) stromelysins 1 and 2 (metalloproteinase-3 and metalloproteinase-10); (iv) matrilysin (MMP-7), enamelysin (MMP-20), macrophage metalloelastase (MMP-12) and MMP-19 (making up the classical metalloproteinases); and (v) membrane-type metalloproteinases (MT-MMP-1 to 4, stromelysin-3, and MMP-11). These metalloproteinases share a common multi-domain structure but are glycosylated to different extents and at different sites. According to sequence alignments, the assembly of these domains might have been an early evolutionary event, followed by diversification.

Collectively, metalloproteinases can degrade all the major components of the extracellular matrix (ECM). The homeostasis of the ECM is controlled by a delicate balance between the synthesis of ECM proteins, the production of ECM-degrading extracellular matrix metalloproteinases, and the presence of metalloproteinase tissue inhibitors.

One family of metalloproteinase inhibitor peptides is the tissue inhibitors of metalloproteinases (TIMPs). The TIMP family is comprised of at least four distinct members (TIMP-1 to 4) that possess 12 conserved cysteine residues and express metalloproteinase inhibitory activity by forming non-covalent complexes with metalloproteinase enzymes. Specifically, TIMPs bind to the highly conserved active zinc-binding site of metalloproteinases in a 1:1 stoichiometry, but can also bind at other domains of metalloproteinase-2.

WO 2010126544 describes the use of mast cell stabilizers to prevent, treat, or mitigate the severity of laminitis. US 20140144109 discloses a boot for treating laminitis in horses wherein the boot has a hoof casing for snugly receiving and supporting the hoof wall of the laminitic hoof and a sole pivotally attached to the hoof casing such that the laminitic hoof may pivot with respect to the sole while the sole is planted on the ground, thereby reducing stress on the inflamed laminae. EP 2497475 describes the use of specific anti-platelet drugs for the treatment and/or prevention of laminitis. However, the prior art in this field has not been able to effectively prevent or cure laminitis.

Provided is a method for treating or preventing a disorder associated with undesirable immune inflammatory activity leading to protease activity in a subject in need thereof, the method comprising administering a therapeutically or prophylactically effective amount of a 21-aminosteroid (also known as a lazaroid), or a therapeutically acceptable salt thereof to the subject.

Also provided is a method for treating or preventing a disorder associated with undesirable immune inflammatory activity leading to protease activity in a subject in need thereof, the method comprising administering to a subject a therapeutically or prophylactically effective amount of a 21-aminosteroid or a therapeutically acceptable salt thereof.

Also provided is a method for treating or preventing laminitis in an ungulate in need thereof, the method comprising administering to the ungulate a therapeutically or prophylactically effective amount of a 21-aminosteroid or a therapeutically acceptable salt thereof.

Also provided is a method for treating or preventing equine chronic lung disease in an ungulate in need thereof, the method comprising administrating to the ungulate a therapeutically or prophylactically effective amount of a 21-aminosteroid or a therapeutically acceptable salt thereof.

Also provided is a method for treating or preventing equine osteoarthritis disease in an ungulate in need thereof, the method comprising administrating to the ungulate a therapeutically or prophylactically effective amount of a 21-aminosteroid or a therapeutically acceptable salt thereof.

Also provided is a method for treating or preventing equine septic joint disease in an ungulate in need thereof, the method comprising administrating to the ungulate a therapeutically or prophylactically effective amount of a 21-aminosteroid or a therapeutically acceptable salt thereof.

Also provided is a method for treating or preventing equine colic disease in an ungulate in need thereof, the method comprising administrating to the ungulate a therapeutically or prophylactically effective amount of a 21-aminosteroid or a therapeutically acceptable salt thereof.

Also provided is a method for treating or preventing equine chronic obstructive pulmonary disease in an ungulate in need thereof, the method comprising administrating to the ungulate a therapeutically or prophylactically effective amount of a 21-aminosteroid or a therapeutically acceptable salt thereof.

Also provided is a method for treating or preventing equine ulcerative colitis disease in an ungulate in need thereof, the method comprising administrating to the ungulate a therapeutically or prophylactically effective of a 21-aminosteroid or a therapeutically acceptable salt thereof.

Also provided is a method for treating or preventing equine Crohn's disease in an ungulate in need thereof, the method comprising administrating to the ungulate a therapeutically or prophylactically effective amount of a 21-aminosteroid or a therapeutically acceptable salt thereof.

Also provided is a method for treating or preventing equine inflammatory bowel disease in an ungulate in need thereof, the method comprising administrating to the ungulate a therapeutically or prophylactically effective amount of a 21-aminosteroid or a therapeutically acceptable salt thereof.

Also provided is a method for preventing or treating a condition associated with a gastrointestinal injury, disease, or ulcer in an animal in need thereof, comprising administering to the animal a therapeutically or prophylactically effective amount of a 21-aminosteroid or a therapeutically acceptable salt thereof.

These and other aspects of the disclosure disclosed herein will be set forth in greater detail as the patent disclosure proceeds.

### BRIEF DESCRIPTION OF THE SEQUENCES

SEQ ID NO:1—Sequence of anti-hemorrhagic peptide (15 amino acids).

SEQ ID NO:2—Sequence of anti-hemorrhagic peptide (10 amino acids).

### DETAILED DESCRIPTION

The inventor has demonstrated that 21-aminosteroids (lazaroids) are useful to inhibit the cell-damaging effects of protease enzymes, inflammatory cytokines, and oxidative radicals produced by these.

In some embodiments, the 21-aminosteroid, or a therapeutically acceptable salt thereof is chosen from a compound of Formula I:

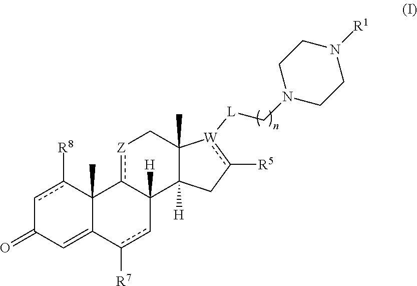

or a therapeutically acceptable salt thereof, wherein
W is chosen from C and CR⁴;
Z is CR⁶;
R¹ is heteroaryl, optionally substituted by one or more R² groups;
each R² is independently chosen from heterocycloalkyl and amino, either of which may be optionally substituted by one or more R³ groups;
each R³ is independently chosen from alkyl and allyl;
R⁴ is chosen from H, hydroxyl, and alkyl;
R⁵ is chosen from H and alkyl;
R⁶ is chosen from H, hydroxyl, and oxo;
R⁷ is chosen from H, halo, and alkyl;
R⁸ is chosen from H and hydroxyl;
L is chosen from —CH₂—, —C(O)—, and —C(CH₃)H—; and
n is chosen from 1, 2, or 3.

In some embodiments, R¹ is chosen from pyridinyl and pyrimidinyl, either of which may be optionally substituted by one or two R² groups.

In some embodiments, R¹ is pyrimidinyl, substituted by one or two R² groups.

In some embodiments, R² is independently chosen from pyrrolidinyl, morpholinyl, diethylamino, amino, dimethylamino, piperazinyl, 4-methyl-1-piperazinyl, piperidinyl, allylamino, and ethylamino.

In some embodiments, each R² is pyrrolidinyl.

In some embodiments, each R³ is independently chosen from C1-C3 alkyl and allyl.

In some embodiments, R⁴ is chosen from H, hydroxyl, and C1-C3 alkyl.

In some embodiments, R⁴ is H.

In some embodiments, R⁵ is H.

In some embodiments, R⁵ is chosen from H and C1-C3 alkyl.

In some embodiments, R⁵ is methyl.

In some embodiments, R⁷ is chosen from H, halo, and C1-C3 alkyl.

In some embodiments, R⁶, R⁷, and R⁸ are H, L is —C(O)—, and n is 1.

The method of claim 1, wherein the 21-aminosteroid is chosen from: U-74389G, tirilazad mesylate, 21-[4-[2-amino-6-(diethylamino)-4-pyrimidinyl]-1-piperazinyl]-17α-hydroxypregna-4,9(11)-diene-3,20-dione,
17α-hydroxy-21-[4-[2,6-bis(dimethylamino)-4-pyrimidinyl]-1-piperazinyl]pregna-4,9(11)-diene-3,20-dione, 21-[4-[2-(diethylamino)-6-(1-pyrrolidinyl)-4-pyrimidinyl]-1-piperazinyl]-17α-hydroxypregna-4,9(11)-diene-3,20-dione,
17α-hydroxy-21-[4-[2-(diethylamino)-6-(4-methyl-1-piperazinyl)(4-pyrimidinyl)]-1-piperazinyl]pregna-4,9(11)-diene-3,20-dione,
17α-hydroxy-21-[4-[2,6-bis(diethylamino)-4-pyrimidinyl]1-piperazinyl]pregna-4,9(11)-diene-3,20-dione,
1α-hydroxy-21-[4-[2-(diethylamino)-6-(1-piperidinyl)-4-pyrimidinyl]-1-piperazinyl]pregna-4,9(11)-diene-3,20-dione,
21-[4-[2,6-bis(diethylamino)4-pyrimidinyl]-1-piperazinyl]-17α-hydroxy-16α-methylpregna-1,4,9(11)-triene-3,20-dione,
17α-hydroxy-21-[4-[2,6-bis(4-methyl-1-piperazinyl)(4-pyrimidinyl)]-1-piperazinyl]pregna-4,9(11)-diene-3,20-dione,
17α-hydroxy-6α-methyl-21[4-[2,6-bis-(1-pyrrolidinyl)-4-pyrimidinyl]-1-piperazinyl]pregna-1,4,9(11)-triene-3,20-dione,
21-[4-[2,6-bis(diethylamino)-4-pyrimidinyl]-1-piperazinyl]-11α,17α-dihydroxypregn-4-ene-3,20-dione,
17α-hydroxy-21-[4-[2,6-bis(diethylamino)-4-pyrimidinyl]-1-piperazinyl]-pregn-4-ene-3,20-dione,
21-[4-[2,6-bis(diethylamino)-4-pyrimidinyl]-1-piperazinyl]-17α-hydroxy-6α-methylpregna-1,4,9(11)-triene-3,20-dione,
17α-hydroxy-21-[4-[2,6-bis(1-pyrrolidinyl)-4-pyrimidinyl]-1-piperazinyl]pregna-4,9(11)-diene-3,20-dione,
21-[4-[2,6-bis(diethylamino)-4-pyrimidinyl]-1-piperazinyl]-11α-hydroxypregn-4-ene-3,20-dione,
21-[4-[2,6-bis(diethylamino)-4-pyrimidinyl]-1-piperazinyl]-11α,17.alpha.-dihydroxypregn-4-ene-3,20-dione,
17α-hydroxy-16α-methyl-21-[4-[2,6-bis-(1-pyrrolidinyl)-4-pyrimidinyl]-1-piperazinyl]pregna-1,4,9(11)-triene-3,20-dione,
17α-hydroxy-21-[4-[2,6-bis(1-pyrrolidinyl)-4-pyrimidinyl]-1-piperazinyl]pregna-1,4,9(11)-triene-3,20-dione,
21-[4-[2,6-bis(diethylamino)-4-pyrimidinyl]-1-piperazinyl]-17α-hydroxypregna-1,4,9(11)-triene-3,20-dione,
21-[4-[4,6-bis(diethylamino)-2-pyrimidinyl]-1-piperazinyl]-17α-hydroxypregna-1,4,9(11)-triene-3,20-dione,
21-[4-[2,6-bis(diethylamino)-4-pyrimidinyl]-1-piperazinyl]-16α-methylpregna-1,4,9(11)-triene-3,20-dione,
21-[4-[2,6-bis(diethylamino)-4-pyrimidinyl]-1-piperazinyl]-11α-hydroxy-16α-methylpregna-1,4-diene-3,20-dione,
21-[4-[2,6-bis(diethylamino)-4-pyrimidinyl]-1-piperazinyl]-16α-methylpregna-1,4-diene,3,20-dione,
16α-methyl-21-[4-[2,6-bis(1-pyrrolidinyl)-4-pyrimidinyl]-1-piperazinyl]pregna-1,4,9(11)-triene-3,20-dione,
11α-hydroxy-16α-methyl-21-[4-[2,6-bis(1-pyrrolidinyl)-4-pyrimidinyl]piperazinyl]pregna-1,4-diene-3,20-dione,
16α-methyl-21-[4-[2,6-bis(1-pyrrolidinyl)-4-pyrimidinyl]-1-piperazinyl]pregna-1,4-diene-3,20-dione,
16α-methyl-21-[4-[2,6-bis(4-morpholino)-4-pyrimidinyl]-1-piperazinyl]pregna-1,4,9(11)-triene-3,20-dione,
11α-hydroxy-16α-methyl-21-[4-[2,6-bis(4-morpholino)-4-pyrimidinyl]-1-piperazinyl]pregna-1,4-diene-3,20-dione,
16α-methyl-21-[4-[2,6-bis(4-morpholino)-4-pyrimidinyl]-1-piperazinyl]pregna-1,4-diene-3,20-dione,
21-[4-[2,6-bis(allylamino)-4-pyrimidinyl]-1-piperazinyl]-16α-methylpregna-1,4,9(11)-triene-3,20-dione,
21-[4-[2,6-bis(allylamino)-4-pyrimidinyl]-1-piperazinyl]-11α-hydroxy-16α-methylpregna-1,4-diene-3,20-dione,
21-[4-[2,6-bis(allylamino)-4-pyrimidinyl]-1-piperazinyl]-16α-methylpregna-1,4-diene-3,20-dione,
21-[4-[2,6-bis(1-pyrrolidinyl)-4-pyrimidinyl]-1-piperazinyl]pregn-4-ene-3,11,20-trione,
21-[4-[2,6-bis(1-pyrrolidinyl)-4-pyrimidinyl]-1-piperazinyl]pregna-4,9(11)-diene-3,20-dione,
21-[4-[2,6-bis(1-pyrrolidinyl)-4-pyrimidinyl]-1-piperazinyl]pregna-1,4-diene-3,20-dione,
21-[4-(2,6-bis(1-pyrrolidinyl)-4-pyrimidinyl)-1-piperazinyl]pregna-4,9(11)-diene-3,20-dione,
21-[4-(2,6-bis(4-morpholino)-4-pyrimidinyl)-1-piperazinyl]-17α-hydroxypregna-4,9(11)-diene-3,20-dione,
21-[4-(2,6-bis(1-pyrrolidinyl)-4-pyrimidinyl)-1-piperazinyl]pregna-4-en-3-one,
21-[4-(2,6-bis(1-pyrrolidinyl)-4-pyrimidinyl)-1-piperazinyl]pregn-4-en-3-one,
16α-methyl-21-[4-[2,6-bis(1-pyrrolidinyl)-4-pyrimidinyl]-1-piperazinyl]pregna-1,4,9(11)-triene-3,20-dione,
21-[4-(2,6-bis(1-pyrrolidinyl)-4-pyrimidinyl)-1-piperazinyl]pregna-1,4,9(11)-triene-3,20-dione,
21-[4-(2,6-bis(1-pyrrolidinyl)-4-pyrimidinyl)-1-piperazinyl]-20-methylpregna-1,4-dien-3-one,
21-[4-(2,6-bis(1-pyrrolidinyl)-4-pyrimidinyl)-1-piperazinyl]pregna-1,4,9(11),16-tetraene-3,20-dione,
21-[4-[2,6-bis(4-morpholino)-4-pyrimidinyl]-1-piperazinyl]pregna-1,4-diene-3,20-dione,
21-[4-[2,6-bis(diethylamino)-4-pyrimidinyl]-1-piperazinyl]-6α-fluoro-17α-hydroxy-160-methylpregna-4,9(11)-diene-3,20-dione,
6α-fluoro-17α-hydroxy-16β-methyl-21-[4-[2,6-bis(1-pyrrolidinyl)-4-pyrimidinyl]-1-piperazinyl]pregna-4,9(11)-diene-3,20-dione,
16α-methyl-21-[4-[2,6-bis(1-pyrrolidinyl)-4-pyrimidinyl]-1-piperazinyl]pregna-1,4-diene-3,20-dione,
21-[4-(2,6-bis(1-pyrrolidinyl)-4-pyrimidinyl)-1-piperazinyl]-16α,17α-dimethylpregna-1,4,9(11)-triene-3,20-dione,
3β-hydroxy-16α-methyl-21-[4-[2,6-bis(1-pyrrolidinyl)-4-pyrimidinyl]-1-piperazinyl]-pregn-5-en-20-one,
16α-methyl-21-[4-[2,6-bis-(1-pyrrolidinyl)-4-pyrimidinyl]-1-piperazinyl]pregna-1,4,6,9(11)-tetraene-3,20-dione,
3β-hydroxy-16α-methyl-21-[4-[2,6-bis(1-pyrrolidinyl)-4-pyrimidinyl]-1-piperazinyl]pregn-5-en-20-one,
16α-methyl-170-(1-oxo-4-[4-[2,6-bis(1-pyrrolidinyl)-4-pyrimidinyl]-1-piperazinyl]butyl)-androsta-4,9(11)-dien-3-one,
21-[4-[5,6-bis(diethylamino)-2-pyridinyl]-1-piperazinyl]-16α-methylpregna-1,4,9(11)-triene-3,20-dione,
21-[4-[3-(ethylamino)-2-pyridinyl]piperazinyl]-16α-methylpregn-1,4,9(11)-triene-3,20-dione,
3,17α-dihydroxy-21-[4-[2,6-bis(1-pyrrolidinyl)-4-pyrimidinyl]-1-piperazinyl]-19-norpregna-1,3,5(10)-trien-20-one,
21′-[4-(2,6-bis(1-pyrrolidinyl)-4-pyrimidinyl)-1-piperazinyl]pregna-1,4,9(11),16-tetraene-3,20-dione,
3α-hydroxy-16α-methyl-21-[4-[2,6-bis(1-pyrrolidinyl)-4-pyrimidinyl]-1-piperazinyl]-5α-pregnan-20-one, and
3β-hydroxy-16α-methyl-21-[4-[2,6-bis(1-pyrrolidinyl)-4-pyrimidinyl]-1-piperazinyl]-5α-pregnan-20-one,
   or a therapeutically acceptable salt thereof.

In some embodiments, the 21-aminosteroid is U-74389G/Methylated Tirilazad or a therapeutically acceptable salt thereof. In some embodiments, the 21-aminosteroid is the maleate salt of Methylated Tirilazad.

In some embodiments, the 21-aminosteroid is Tirilazad or a therapeutically acceptable salt of Tirilazad. In some embodiments, the 21-aminosteroid is the mesylate salt of Tirilazad.

One of skill in the art would understand that a 21-aminosteroid or other drug or compound described herein may be known in the art by alternate names; these alternate names are intended to be encompassed with the scope of the present disclosure.

The 21-aminosteroid may be administered alone, but will typically be administered as a pharmaceutical composition, which will generally comprise a suitable pharmaceutical excipient, diluent, or carrier selected depending on the intended route of administration, which is usually intravenous (IV).

The 21-aminosteroid may be provided at a concentration ranging from about 0.01 μg/ml to about 100 mg/ml in the formulation. Typically, 21-aminosteroid is present at a concentration ranging from about 0.1 μg/ml to about 1000 μg/ml. More typically, the 21-aminosteroid is present at a concentration ranging from about 1 μg/ml to 500 μg/ml.

In some embodiments, a 21-aminosteroid described herein may be administered in combination with an anti-hemorrhagic peptide. In some embodiments, the anti-hemorrhagic peptide may be obtained from opossum serum or cotton rat, or may be a recombinant form thereof. In some embodiments, the anti-hemorrhagic peptide may be artificially produced or synthesized. In some embodiments, an anti-hemorrhagic peptide described herein may have an amino acid sequence of any of the following, or combinations thereof:
Phe-Leu-His—Peptide 1
Trp-Leu-Phe—Peptide 2
Trp-Leu-Try—Peptide 3
Trp-Leu-Arg—Peptide 4
Trp-Leu-His—Peptide 5
Phe-Leu-Phe—Peptide 6
Phe-Leu-Try—Peptide 7
Phe-Leu-Arg=Peptide 8

In some embodiments, the peptide is coupled to hydroxamate.

In some embodiments, the peptide comprises, consists of, or consists essentially of one of the following sequences:

(SEQ ID NO: 1)

Leu-Lys-Ala-Met-Asp-Pro-Thr-Pro-Pro-Leu-Trp-Ile-
Lys-Thr-Glu

(SEQ ID NO: 2)

Leu-Lys-Ala-Met-Asp-Pro-Thr-Pro-Pro-Leu.

In some embodiments, an anti-hemorrhagic peptide described herein has an amino acid sequence that has at least 85%, 90%, 95%, 96%, 97%, 98%, or 99% sequence identity with SEQ ID NO:1 or SEQ ID NO:2.

In some embodiments, an anti-hemorrhagic peptide described herein may be administered simultaneously with, sequentially with, or separately to the 21-aminosteroid.

Pharmaceutical compositions may be adapted for administration in any suitable manner. The composition may be adapted for intravenous (IV) or depot (e.g., slow release) administration. The composition may be in injectable or suppository form, or may be formulated in a gel to make application to wound surfaces more convenient. In some embodiments, delivery routes are intravenous or intramuscular depot administration.

Methods and pharmaceutical carriers for preparation of pharmaceutical compositions, including compositions for intravenous administration, are well-known in the art.

Compositions may be formulated so that they are suitable for intravenous administration.

In addition to the ingredients particularly mentioned above, the compositions may include other agents conventional in the art having regard to the type of therapeutic in question, for example, those suitable for intravenous administration.

The composition may have at least one further active ingredient selected from antibiotics, anti-inflammatories, antiseptics, and other agents, e.g. anesthetics. The compositions may have other molecules associated therewith to aid releasability, stability, solubility, activity and/or association with wound healing, including carriers, solubilizing agents, and growth factors. The composition may also include one or more secondary therapeutic agents for treatment of the disorder in question, such as laminitis.

Laminitis can be stimulated by bacterial and/or viral infection, or by a gastrointestinal injury, disease, infection, or ulcer. Laminitis may be stimulated from a disease or condition such as equine chronic lung disease, equine osteoarthritis disease, equine septic joint disease, equine colic, equine chronic obstructive pulmonary disease, equine joint disease, equine ulcerative colitis, equine Crohn's disease, or equine inflammatory bowel disease. All of these conditions can be prevented or treated by administering adequate amounts of selected 21-aminosteroid salt.

A subject appropriate for treatment as described herein may be a mammal, such as an ungulate, in particular a hoofed ungulate. In some embodiments, the subject may be an animal from a group such equidae, bovinae, suidae, deer, ovis, and capra. Typically, the treated subject is a horse or dairy cow of economic importance.

A 21-aminosteroid described herein may be injected intravenously into the ungulate, for example, the hoof. The ungulate may be suspected of developing or may have the sequela of laminitis at the time of administration. Administration of the 21-aminosteroid may prevent laminar detachment. A 21-aminosteroid may be administered into the flexor digitorum profundus muscle or into the blood supply of a limb of the ungulate.

Also provided is a method for preventing or treating a condition associated with a gastrointestinal injury, disease, or ulcer, the method including administering to the animal in need thereof an effective amount of a 21-aminosteroid salt as described herein.

In some embodiments, the concentration of the 21-aminosteroid present in a formulation suitable for intravenous administration should range from about 0.1 μg/ml to about 10 mg/ml, including 0.1 μg/ml, 0.2 μg/ml, 0.3 μg/ml, 0.4 μg/ml, 0.5 μg/ml, 0.6 μg/ml, 0.7 μg/ml, 0.8 μg/ml, 0.9 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 65 μg/ml, 70 μg/ml, 75 μg/ml, 80 μg/ml, 85 μg/ml, 90 μg/ml, 95 μg/ml, 100 μg/ml, 150 μg/ml, 200 μg/ml, 250 μg/ml, 300 μg/ml, 350 μg/ml, 400 μg/ml, 450 μg/ml, 500 μg/ml, 550 μg/ml, 600 μg/ml, 650 μg/ml, 700 μg/ml, 750 μg/ml, 800 μg/ml, 850 μg/ml, 900 μg/ml, 950 μg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, or any particular concentrations between.

The composition may be administered at any appropriate time, including prior to, during, or after the disorder has become evident. Typically, two or more doses may be administered over time, such as 2, 3, 4, 5, 6, 7, 8, 9, 10, 12, or more doses. The composition may be administered for any period of time deemed appropriate to alleviate or prevent symptoms of laminitis, such as 1 day, 2 days, 3 days, 4 days, 5 days, 6 days, 7 days, 8 days, 9 days, 10 days, 11 days, 12 days, 13 days, 14 days, or more.

For all methods of treatment described herein, the daily dosage can be routinely determined. Generally, the dosage will vary according to the age, weight, and response of the individual, as well as the severity of the symptoms. In general, a suitable dose of a 21-aminosteroid described herein will be in the range of about 0.1 μg to about 100 mg per kilogram body weight of the subject or recipient per day, such as including, but not limited to, 0.1 μg, 0.2 μg, 0.3 μg, 0.4 μg, 0.5 μg, 0.6 μg, 0.7 μg, 0.8 μg, 0.9 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, or any particular concentrations between. In some embodiments, a suitable dose of a 21-aminosteroid will be in the range of about 1 μg to about 50 mg per kilogram body weight per day.

Also provided is a method for treating or preventing a disorder associated with undesirable protease activity in a subject in need thereof, the method comprising administering to the subject in need thereof an effective amount of a 21-aminosteroid salt as described herein in combination with an effective amount of an anti-hemorrhagic peptide described herein.

Also provided is a method for preventing or treating a condition associated with a gastrointestinal injury, disease, or ulcer, the method including administering to the subject in need thereof an effective amount of a 21-aminosteroid salt as described herein in combination with an effective amount of an anti-hemorrhagic peptide described herein.

In certain embodiments, the anti-hemorrhagic peptide is obtained from opossum serum or cotton rat, or is a recombinant form thereof. In certain embodiments, the anti-hemorrhagic peptide is selected from a peptide disclosed herein, such as SEQ ID NO:1 or SEQ ID NO:2, the following or SEQ ID NO:2. In some embodiments, an anti-hemorrhagic peptide may be one or more of peptides 1-8 disclosed herein. In some embodiments, any combinations of these peptides may be used as described herein.

In some embodiments, the concentration of an anti-hemorrhagic peptide described herein may be in a dose range of from about 0.1 μg/ml to about 10 mg/ml, including, but not limited to, 0.1 μg, 0.2 μg, 0.3 μg, 0.4 μg, 0.5 μg, 0.6 μg, 0.7 μg, 0.8 μg, 0.9 μg, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1 mg, 2 mg, 3 mg, 4 mg, 5 mg, 6 mg, 7 mg, 8 mg, 9 mg, 10 mg, or any particular concentrations between.

In some embodiments, an anti-hemorrhagic peptide described herein may be administered at any appropriate time, including prior to, during, or after the disorder has become evident. Any number of doses of an anti-hemorrhagic peptide may be administered as deemed appropriate. Typically, two or more doses may be administered over time.

For all methods of treatment described herein, the daily dosage for an anti-hemorrhagic peptide can be routinely determined by the attending physician or veterinarian. Generally, the dosage will vary according to the age, weight, and response of the individual patient or subject, as well as the severity of the patient's symptoms. In general, a suitable dose of an anti-hemorrhagic peptide described herein will be in the range of about 0.1 μg to about 100 mg per kilogram body weight of the subject or recipient per day, preferably in the range of about 1 μg to about 50 mg per kilogram body weight per day. However, the dose will also depend on the formulation, purity, and concentration of the components of the formulation used, e.g., the 21-aminosteroid salt and/or the anti-hemorrhagic peptide

A disorder appropriate for treatment as described herein can be a dental or oral wound; peptic ulceration of the duodenum, stomach or esophagus; inflammatory bowel disease; an ulcer associated with stress conditions; damage to the lining of the alimentary tract; inadequate gut function or damage to the gut associated with prematurity; a diarrheal condition; a food intolerance; a cancer of the gastrointestinal tract; surgically induced damage to the gut; damage due to esophageal reflux; a condition associated with loss of gut barrier function; a congenital condition resulting in inadequate gastrointestinal function or damage; or an autoimmune disease that affects the gut.

### Definitions

As used herein, the terms below have the meanings indicated.

Tirilazad refers to 16α-methyl-21-[4-[2,6-bis(1-pyrrolidinyl)-4-pyrimidinyl]-1-piperazinyl]pregna-1,4,9(11)-triene-3,20-dione, which has the following structure.

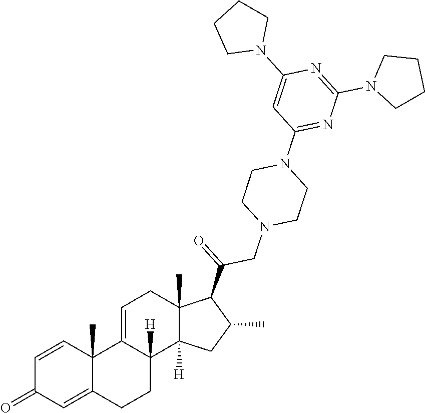

Methylated tirilazad or U-74389G refers to 21-[4-(2,6-bis(1-pyrrolidinyl)-4-pyrimidinyl)-1-piperazinyl]pregna-1,4,9(11)-triene-3,20-dione, which has the following structure:

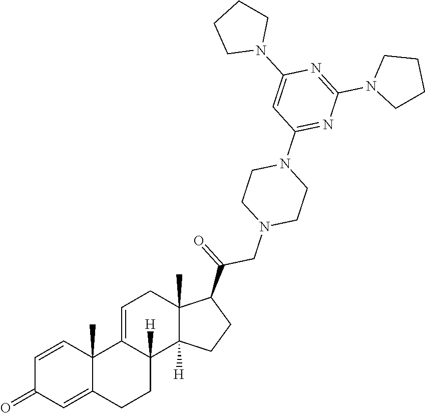

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms.

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “halo,” or “halogen,” as used herein, alone or in combination, refers to fluorine, chlorine, bromine, or iodine.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. The heterocycle groups may be optionally substituted unless specifically prohibited.

The term “hydroxy,” as used herein, alone or in combination, refers to —OH.

The term “oxo,” as used herein, alone or in combination, refers to ═O.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

The term “optionally substituted” means the anteceding group may be substituted or unsubstituted.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “disease” as used herein is intended to be generally synonymous, and is used interchangeably with, the terms “disorder,” “syndrome,” and “condition” (as in medical condition), in that all reflect an abnormal condition of the animal body or of one of its parts that impairs normal functioning, is typically manifested by distinguishing signs and symptoms, and causes the animal to have a reduced duration or quality of life.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

The compounds disclosed herein can exist as therapeutically acceptable (e.g., biologically effective) salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion.

As used herein, a “therapeutically effective amount” of a compound, drug, or other agent refers to an amount that is sufficient to generate a desired therapeutic response, such as reduce, eliminate, or measurably alter outward signs or symptoms of a condition or disease described herein. For instance, as described herein, a therapeutically effective amount may be an amount necessary to reduce, eliminate, ameliorate, or otherwise improve clinical or histopathological signs and/or symptoms of laminitis described herein. Histopathological symptoms of laminitis may include, but are not limited to, separation of the basement membrane, pyknosis or karyopyknosis, and presence of cytoplasmic and nuclear alteration in epidermal cells. A therapeutically effective amount may also be sufficient to reduce the Obel score of an animal, e.g., a horse, indicating an improvement of signs/symptoms of laminitis. A therapeutically effective amount is also an amount required to at least partly attain a desired effect, i.e., to alleviate or remove the symptoms associated with undesirable immune inflammatory activity leading to protease activity, or alternatively to delay the onset of, inhibit the progression of, or halt altogether, the onset or progression of the undesirable immune inflammatory activity leading to protease activity. Preferably, the term “therapeutically effective amount” as used herein means amount sufficient to elicit a statistically significant response at a 95% confidence level. Such amounts will depend, of course, on the particular condition being treated, the severity of the condition, and individual subject parameters, including age, physical condition, size, weight, and other concurrent treatment, and will be at the discretion of the attending veterinary person. These factors are well known to those of ordinary skill in the art, and can be addressed with no more than routine experimentation. It is generally preferred that a minimum effective dose be determined according to sound veterinary judgment.

An effective amount may be a prophylactically effective amount, which is an amount that prevents one or more signs or symptoms of a particular disease or condition from developing. A prophylactically effective amount is an amount that prevents the development of histopathological signs/symptoms of laminitis, or otherwise provides a protective effect to the subject.

As used herein, the term “treatment” refers to any regime that can benefit a subject. References herein to “therapeutic” and “prophylactic” treatment are to be considered in their broadest context. The term “therapeutic” does not necessarily imply that a subject is treated until total recovery. Similarly, “prophylactic” does not necessarily mean that the subject will not eventually contract a disease condition. Accordingly, therapeutic and prophylactic treatment includes amelioration of the symptoms of a particular condition or preventing or otherwise reducing the risk of developing a particular condition.

As used herein, “treating or preventing laminitis” refers to a reduction, improvement, lessening, elimination, reversal, or prevention of the development of signs and/or symptoms of a disease described herein. For example, treating laminitis refers to an improvement or reduction in clinical signs or symptoms of laminitis, e.g., difficulty walking, improvement in gait or turning, improved ability to lift the affected leg, or a reduction in an Obel score for an evaluated animal. Preventing laminitis refers to the prevention of development of a clinical or histopathological indication of laminitis, e.g., separation of the basement membrane, following administration of tirilazad or a therapeutically acceptable salt thereof. Prophylactic administration of tirilazad may be preferred in certain instances where animals are subjected to conditions that are favorable for developing laminitis as described herein. In such instances, administration of tirilazad or another compound disclosed herein may prevent the development of cellular degeneration and other histopathological indications of laminitis, thereby preventing laminitis or development of clinical signs of disease in the animal.

### EXAMPLES

### Example 1—Clinical Study of Tirilazad for Treatment of Acute Laminitis in Horses

A clinical study was carried out to evaluate the potential prophylactic and/or therapeutic properties of a 21-aminosteroid salt when employed as an intravenous treatment in a carbohydrate overload model of acute laminitis in horses.

### Protocol for Clinical Study

### Study Objective

The objective of the study is to evaluate the ability of a 21-aminosteroid (Tirilazad) to prevent or ameliorate clinical signs and histopathologic changes of acute laminitis. The investigational product will be administered at different time points IV after implementation of an oligofructose overload model shown to cause acute laminitis in mature horses.

TABLE 1
Schedule of Events

| Study Day | Activity |
|---|---|
| Prior to Day −10 | Acquisition of nine or more healthy, mature horses (3 to 7 years) |
| | as candidates. Testing to rule out PPID or EMS. |
| −10 | Begin acclimation |
| −10 to −1 | Once Daily Health Observations |
| Once between −10 | Physical examination, body weights; lameness examinations and |
| and −4 | radiography of both forefeet |
| −3, −2, | Oligofructose regimen with 1 gram/kg orally once daily added to |
| −1 | feed |
| 0 | Pre-induction lameness exam (with video record); serum sample |
| | for baseline ELISA. Complete oligofructose regimen with 10 |
| | grams/kg administered by nasogastric tube; begin clinical |
| | observations, temperature measurement and lameness |
| | monitoring q6h until development of diarrhea. |
| 0 or 1 | Continue clinical observations, temperature and lameness |
| | evaluations q6h. All groups: start intravenous fluids upon first |
| | signs of diarrhea; PCV and TP at least q4h and regulation of |
| | fluids to maintain normal hydration. |
| As needed | (Treatment of Group 2 horses upon onset of pyrexia and/or |
| | diarrhea - prior to any Obel lameness score ≥ 1). Video record of |
| | lameness exam for any horse achieving Obel ≥1. Treatment of |
| | Group 3 horses upon achieving Obel lameness score ≥1 |
| As needed | Continue fluid therapy to maintain hydration |
| As needed | Euthanasia and lamina harvest with Obel lameness score of 3 or |
| | greater. Final observations and temperature measurement prior |
| | to euthanasia. |
| As needed | Collect serum sample for ELISA. Euthanasia and lamina harvest |
| | if Obel lameness score ≤2 for 72 hours after induction. Clinical |
| | observations, temperature measurement, lameness monitoring, |
| | and lameness video prior to euthanasia. |
| | Trial terminates upon last necropsy |

### Treatment Groups

The study will be performed with three treatment groups comprised of three mature horses each.

TABLE 2
Study Groups

| | | Number |
|---|---|---|
| | | of |
| Group | Treatment | Horses |
| 1 | Carbohydrate overload induction - | 3 |
| | placebo | |
| 2 | Carbohydrate overload induction - | 3 |
| | treatment at first | |
| | signs of pyrexia and/or diarrhea | |
| 3 | Carbohydrate overload induction - | 3 |
| | treatment upon | |
| | achievement of Obel lameness | |
| | score ≥1 | |

### Experimental Design

This is an unmasked, controlled, randomized, laboratory efficacy study conducted at a single site. Mature, healthy horses with no evidence of prior laminitis episodes will be subjected to a carbohydrate overload induction model for acute laminitis. Within 12 to 24 hours after the final induction step, subjects are expected to become febrile and develop profuse diarrhea accompanied by dehydration and shock. The dramatic physiologic disruptions induced by carbohydrate overload frequently culminate in acute laminitis. All affected animals will be treated with intravenous electrolyte solutions for volume replacement and maintenance of physiologic homeostasis (volume replacement may occur simultaneously with specific treatments in Groups 2 and 3). Three horses (Group 1) will receive 6 mL/kg bodyweight of 0.9% saline solution (placebo) immediately after onset of pyrexia and/or diarrhea. Thereafter, Group 1 horses will receive fluid therapy for dehydration and shock, but no specific treatments will be administered for laminitis. Three horses (Group 2) will receive the experimental treatment of IV 21-aminosteroid immediately after the onset of pyrexia and/or diarrhea. Three remaining horses (Group 3) will receive the experimental treatment of IV 21-aminosteroid upon achievement of an Obel lameness score ≥1.

After completion of the final induction step (designated Time Zero or T.0), rectal temperatures and heart rates will be measured, mucous membranes will be evaluated for capillary refill time (CRT), and Obel lameness scores will be assigned at 6-hour intervals. Beginning with the onset of diarrhea, intravenous fluids will be administered and packed cell volume (PCV) will be measured at 2-hr intervals. Fluid administration will be titrated to maintain hydration within 4 to 6% of normal.

The humane endpoint for this study is an Obel lameness score of “3”. As soon as a score of “3” is recorded, final measurements and samples will be collected, and the horse will be promptly and humanely euthanatized. After death, both forefeet will be disarticulated and the hooves will be sectioned. Specific measurements will be recorded, and representative samples of hoof lamina will be harvested and preserved for histopathologic analysis.

Any horse that does not achieve an Obel score of “3” will be maintained on study for a maximum of 72 hours after induction. After that interval, the horse will be necropsied and its hooves processed as described previously.

### Randomization and Allocation

Horses meeting the inclusion criteria (see sections 9.2 and 9.3) will be ranked by decreasing body weight. Each three consecutively-ranked horses will comprise a replicate. Within each replicate, one horse will be assigned randomly to one of the three treatment groups presented in Table 1. Each treatment group will be represented once within a replicate.

### Test Animals

Horses are the target species for use of the biological product to be evaluated. This study will utilize mature, random-source, light saddle breed horses, females or neutered males, and 3 to 7 years of age at the time of induction. Candidate horses must have Obel lameness scores of “0”, be sound by hoof tester examination, and exhibit no radiographic evidence of prior laminitic episodes (i.e., no ventral deviation of the third phalanx). Candidate horses will be healthy, as determined by clinical health observations and physical examination during the acclimation period and prior to initiation of the carbohydrate overload induction regimen. Pre-enrollment testing of candidates will be conducted to rule out PPID (Cushing's Disease) and Equine Metabolic Syndrome (insulin resistance). Candidates must have a body condition score of ≥3 to ≤7. Female horses may not be pregnant or lactating.

At least nine candidate horses will be received at the site to begin acclimation, and any that meet inclusion criteria will be enrolled. Additional candidates may be evaluated, but ultimately, only nine will be enrolled in the study.

Horses will be derived from the resident, facility herd or purchased from a commercial livestock vendor.

All horses will be uniquely identified by a numbered neck band and by a complete physical description in the study record.

A candidate horse will be eligible for enrollment if it meets all of the following criteria:
It conforms to the animal description in section 8.1.1 (age, gender, class, physiologic status).
It has no significant health abnormalities, based on historical daily clinical health observations and physical examination during acclimation prior to allocation and carbohydrate overload induction.
It has no diagnostic evidence of concurrent PPID or EMS
It has a Body Condition Score ≥4 and ≤7.
It has an Obel score of “0” and a hoof tester score of “0” for both forefeet
It has no radiographic evidence of prior laminitic episodes.
It is tractable and cooperative with study activities
It received no treatment during the acclimation period with corticosteroids or non-steroidal anti-inflammatory drugs (NSAIDs).

A candidate will be excluded from enrollment if:
It does not conform to inclusion criteria.
It exhibits complicating disease conditions that may interfere with or prevent the evaluations and analyses in this study
It has diagnostic evidence of concurrent PPID or EMS.
It is unsound, as determined by hoof tester or Obel lameness criteria
It exhibits radiographic evidence of prior laminitic episodes.
It has been treated within recent history with corticosteroids or non-steroidal, anti-inflammatory drugs
It has a body condition score (BCS)<4 or >7. # It is fractious or uncooperative with study activities.
It received treatment with corticosteroids or NSAIDS during the acclimation period

### Duration

Horses new to the facility will be present for at least seven days prior to the start of formal acclimation. Horses will be acclimated to the facility for at least seven days prior to initiation of the carbohydrate overload induction regimen. During the acclimation period, feed, water, housing, management, and environmental conditions will simulate those expected during the study.

Medication and/or Vaccination During Acclimation Period

Candidate horses may be treated with approved pharmaceutical products prior to initiation of the acclimation period, but no medicinal products may be administered to subjects from the start of acclimation until completion of the study. Certain prior treatments are proscribed, as described in Section 8.2 and Section 8.3.

If any pharmaceutical product is indicated for treatment of pain, trauma, or spontaneous medical conditions, these may be given for humane reasons, but only after consultation with, and approval by, the Clinical Investigator and/or the Sponsor Representative (see section 8.14.2). All concurrent medications or therapies will be recorded in the study file and mentioned in the final report.

### Masking of Study

Masking will not be implemented in the study. Testing personnel will be aware of treatment group assignments for the various animals.

Similarly, the veterinary histopathologist will be aware of group assignments when examining tissues, consistent with a consensus statement by the Society for Toxicologic Pathology (Crissman et al. Best Practices Guideline: Toxicologic Histopathology. Toxicologic Pathology 32:126-131, 2004).

### Housing Enclosures

All horses acquired for the study will be housed in individual stalls measuring ˜4 m×˜4 m floor area×˜1.7 m high. Stalls will be constructed of portable metal panels. Flooring will be concrete, covered by rubber mats and bedded with pine or hardwood sawdust and shavings.

Horses will remain in their assigned stalls continuously, unless removed temporarily to facilitate cleaning activities or to conduct protocol activities (e.g., body weights, lameness examinations). Each stall is equipped with feeding and watering equipment, and feces are removed from the stalls daily. Soiled bedding is replaced as necessary, usually about once weekly during acclimation, and at least daily during the overload induction phase. Facility details will be described and documented in the study record.

Overhead incandescent lighting is available to provide illumination during late p.m. and early a.m. activities.

The equine housing facility is under roof, but subjects are otherwise exposed to ambient environmental conditions. Climatic conditions (minimum and maximum temperature and relative humidity) will be monitored electronically on a constant basis, and daily minima and maxima will be recorded manually on a data capture form customized for the specific study.

Each stall is equipped with a combination concentrate/hay feeder designed to offer both dietary components simultaneously. Feeders are checked daily and cleaned if necessary.

Facility water is supplied by a local utility. Fresh water is available ad libitum, supplied in two buckets ≥16 L in volume. Buckets are cleaned once daily and filled at least twice daily.

### Feeding

Horses will be offered a commercial horse concentrate (Co-Op #327; 11% protein) in quantities comprising 0.5% of body weight daily, divided into equal portions and offered a.m. and p.m. A feed label will be added to the study record. Horses will also be provided with grass hay at 1.5% of body weight daily, also divided into equal portions offered a.m. and p.m.

### Physical Examinations

A qualified veterinarian will conduct a physical examination during the week of acclimation (between Days −10 and −4). The examination will evaluate the physiological status of each animal by systems, including rectal temperature, eyes, cardiovascular system, respiratory system, gastrointestinal and genitourinary systems, skin and hair coat, neurologic and musculoskeletal function, and overall physical condition. Findings for individual horses will be recorded on the Physical Examination Record.

A Body Condition Score (BCS) will be assigned to each candidate during the physical examination. Scores will range from 1 to 9, and are based on the Henneke system (Henneke et al. Relationship between condition score, physical measurements and body fat percentage in mares. Equine Veterinary Journal 15:371-372, 1983).

### Body Weight

Each candidate will be weighed once between Days −10 to −4. Relevant body weights will be used to calculate appropriate quantities of oligofructose for the carbohydrate overload induction model.

Body weights will be measured with a scale that has been certified by a commercial service within 6 months before the start of the study. Prior to weighing the first animal, and again after weighing the last animal, the accuracy of the scale will be verified with standard weights ranging from 45.4 kg (100 lbs) to 364 kg (800 lbs). Body weights will be measured to the nearest kg and recorded on the Body Weight Record.

### Lameness Examinations

Prior to enrollment, each horse will be assessed for lameness, as described in facility SOP LAM-FD-2.2. Each horse will be assessed at a walk and at a trot (if possible) to assign baseline Obel scores for each forefoot.

Guidelines for Obel lameness scoring are as follows:

Grade 0: No lameness observed at a walk or trot, even on hard surfaces.

Grade 1: The horse may alternately lift its feet, but no lameness is observable at a walk. The horse may have a short, stilted gait when trotting in a straight line on a hard surface, and turns carefully at a walk.

Grade 2: Moves with a stiff gait at the walk. The horse may have a short, stilted gait at a trot on a hard surface. Turns with great difficulty. A foot can be lifted off the ground without great difficulty.

Grade 3: Reluctant to move at a walk on any surface. It is difficult to lift a limb. The horse may be almost non-weight bearing on one limb.

Grade 4: The animal will not move, and is particularly reluctant to move from a soft to a hard surface. It is almost impossible to lift a limb.

Prior to enrollment, horses will be evaluated for foot pain using hoof testers. The hoof testers will be applied in a systemic manner to the entire sole, frog region and hoof wall to test for sensitivity/pain. A hoof tester score of “0” for both forefeet is required to be eligible for enrollment.

Guidelines for hoof tester score are as follows:
0=No pain
1=Mild pain is noted
2=Moderate pain is noted
3=Highly reactive to pain
4=Unable to lift leg

Prior to enrollment, lateral radiographs of both forefeet of each horse will be recorded and examined for evidence of prior laminitis, defined as ventral rotation of the third phalanx (P3) in the lateral view. A written interpretation of each horse's radiographs will be included in the study record.

### Clinical Observations

Clinical health observations will be recorded once daily from Day −10 to the final day of enrollment. The parameters to be observed include general health, appetite, attitude and fecal consistency (Daily Health Observation Record). Findings will be recorded as “normal” or “abnormal”, with further characterization in the study record of any abnormal observation.

At ˜6-hour intervals (±30 minutes) after administration of the final step of the oligofructose model, general health observations will be conducted, along with measurement of rectal temperature, heart rate, and assessment of capillary refill time. Observations will be recorded on Data Capture Forms specifically created for the study, and abnormal observations will be further characterized in the study record.

Beginning when diarrhea is observed, a venous blood sample will be collected every 2 hours for measurement of packed cell volume and total protein concentration. Heart rate, CRT, and total protein concentration will be used to assess dehydration as per ETCR SOP LAM-FD-1.x. Fluid therapy will be initiated at the discretion of the veterinarian or when percentage dehydration achieves 6% or greater.

After administration of the experimental treatment (Groups 2 and 3), any abnormal health observations will qualify as Adverse Events (AE). Within 24 hours after detection of a serious AE, the Clinical Investigator will report the AE to the Sponsor Monitor, and the event will be documented on the Adverse Event Record. The Adverse Event Record will categorize the severity of the abnormal observation, and the reporting veterinarian will speculate as to the relationship of the AE to experimental treatment as follows:

Magnitude of Adverse Event

| Score | Description |
|---|---|
| 1 = Mild | Little or no discomfort. |
| | Signs intermittent or continuous. |
| | Baseline functions unhindered. |
| | Not significantly hazardous to overall health. |
| | Drug therapy and/or clinical procedure not |
| | necessary. |
| 2 = Moderate | Some discomfort. |
| | Signs intermittent or continuous. |
| | Baseline functions moderately hindered. |
| | Not significantly hazardous to overall health. |
| | Drug therapy and/or clinical procedure may be |
| | necessary. |
| 3 = Severe | Severe discomfort. |
| | Signs intermittent or continuous. |
| | Baseline functions severely hindered or prevented. |
| | Significantly hazardous to overall health. |
| | Drug therapy and/or clinical procedure imperative. |

TABLE 3
Magnitude of Adverse Event and Relationship to Experimental Treatment
Relationship of AE to Experimental Treatment

| Score | Description |
|---|---|
| 1 = Unknown | Unknown |
| 2 = Unlikely | Unlikely since AE is clearly pre-existing |
| | or caused by specific |
| | extraneous event, with no other causative |
| | factor evident. |
| 3 = Possible | Possible based on type, time course, and |
| | relationship of AE to |
| | dosing and external events. |
| 4 = Probable | Probable based on type, time course, and |
| | relationship of AE to |
| | dosing and external events. |

### Feed and Water Consumption

Feed and hay will be provided twice daily in weighed quantities. Appetite will be characterized as:
0—consumed <25% of hay/grain
1—consumed 25-75% of hay/grain
2—consumed 75-100% of hay/grain.

Water will be provided in two, 16-L buckets per horse. Water consumption will be measured in ⅛ bucket (i.e., 2-L) increments and recorded twice daily prior to re-filling of the respective water buckets.

### Analytical Methods

The oligofructose overload model of inducing laminitis frequently causes severe diarrhea, so hydration will be monitored beginning at onset of diarrhea and repeated thereafter at ˜2-hr intervals for measurement of packed cell volume (PCV; hematocrit). Packed Cell Volume will be measured by methods described in facility SOP GN-LB-11.3.

Total protein will be measured by examining the plasma portion of a blood column in the microhematocrit tubes described in section 8.13.1. Total protein will be measured with an optical refractometer, as described in SOP GN-LB-11.3. The total protein concentration will be captured in the study record for every interval at which a blood sample is collected for measurement of PCV.

A serum sample (marbled red top tube; 9.5 mL draw) will be collected from each enrolled horse on Day 0 prior to induction, and again just prior to euthanasia. Serum will be harvested from each sample and stored frozen. Serum samples will be shipped to an external laboratory for measurement of camelid antibodies by a proprietary ELISA. Methods and results will be described in a separate report to be prepared by the analytical laboratory.

Removal of Subject(s) from the Study

This protocol seeks to balance the need to generate relevant efficacy data with humane considerations. As such, horses experiencing adverse events, whether or not related to the test article, may receive veterinary care as medically appropriate and under the parameters described above.

A participating horse may be removed from the study if it is determined that:
It is uncooperative with study procedures.
It encounters a serious adverse reaction, injury, or illness necessitating treatment with contraindicated, concomitant medications (see section above) or dictating immediate removal for humane reasons.
It dies spontaneously or is euthanatized.

A horse will be removed from the study if any of the indicated removal criteria apply. The Clinical Investigator will consult with the Sponsor whenever possible prior to removing a horse from the study. However, the final decision whether to remove a horse from the study will rest with the Clinical Investigator. The Clinical Investigator will document the horse's identity, the date of the removal, the reason for the removal, and the fate of the animal. Data generated by removed animals up to the point of removal will be included in study analyses.

Horses withdrawn from the study after being dosed with investigational product will be subject to euthanasia and necropsy, as described in below.

### Induction of Acute Laminitis

### Pre-Induction Dietary Regimen

On Days −3 to −1, one gram of oligofructose (BENEO® 95; Orafti) will be added to the a.m. basal ration of each scheduled candidate. Preparation and administration will be documented in the study record.

### Induction of Acute Laminitis

On Day 0, 10 grams of oligofructose (BENEO 95; Orafti) per kg of body weight will be dissolved in ≥4 liters of tepid tap water. The oligofructose solution will be administered to horses via nasogastric tube. This event will be termed “Time 0” or “T.0”. Preparation and administration will be documented in the study record.

### Euthanasia

Horses will be sedated with xylazine or medetomidine and humanely euthanatized in compliance with recommendations of the 2013 AVMA Guidelines for Euthanasia. Specifically, horses will be dropped with a captive bolt stunner and exsanguinated, as described in facility SOP EQ-NX-1.4. Relevant procedures will be documented on the Equine Euthanasia Record.

### Investigational Product

Chemical name
Tirilazad (21-aminosteroid)
Trade name
BT-002
Active ingredients
U-74389G. Methylated Tirilazad
Dosing form
The investigational product will be formulated for IV administration

### Example 2—In Vitro Study of Tirilazad to Prevent Cellular Effects of Laminitis

An in vitro study was conducted to analyze the protective effects of tirilazad mesylate on cellular degeneration as a result of Bothrops alternatus snake venom as a model for laminitis. Explant tissue samples were obtained from the dermoepidermal junction of the hoof of the right forelimb under nervous system block without the need for euthanasia.

Under sterile conditions, laminar cuts (mini-explants) were made of each explant and the tissue washed with physiological solution. The mini-explants were pre-incubated for 5 hours with tirilazad and then for 24 hours with Bothrops venom. Culture medium was as follows: Dulbecco's minimum essential medium (DMEM, GIBCO-Invitrogen) with 10% heat-inactivated fetal bovine serum (FBS, GIBCO-Invitrogen), L-Glutamine (29.2 mg/mL), Penicillin (10,000 units/mL), and Streptomycin (10,000 μg/mL, GIBCO-Invitrogen) as antibiotics.

Histopathology: Tissues were processed with classical histological techniques, e.g., cutting by microtome (Minot) and stained with hematoxylin-eosin (H-E) and Periodic Acid Schiff (PAS) stains.

Table 4 demonstrates the results from this experiment obtained using a Car Zeiss Primo Star optical microscope.

The tissue incubated only in the culture medium showed no histological alteration.

Tissue incubated with Bothrops alternatus venom at a dose of 75 μg/ml showed intense separation of the basement membrane and alteration of epidermal cells.

Tissue incubated with tirilazad at a dose of 1 μM did not show separation of the basement membrane, and epidermal cells were normal and showed preserved cytoplasmic and nuclear structure.

Tissue incubated with 1 μM tirilazad and 75 μg/ml Bothrops alternatus venom did not show separation of the basement membrane, although intense presence of altered epidermal cells with nuclear pyknosis was observed.

Tissue incubated with 3 μM tirilazad did not show separation of the basement membrane, although slight epidermal cytoplasmic and nuclear alteration was observed.

Incubation of tissue with 3 μM tirilazad and 75 μg/ml Bothrops alternatus venom showed slight separation of the basement membrane and intense presence of altered epidermal cells, with nuclear pyknosis predominating.

Tissue incubated with 10 μM tirilazad did not show separation of the basement membrane, and epidermal cells were normal and showed preserved cytoplasmic and nuclear structure, similar to tissue incubated with 1 μM tirilazad.

Tissue incubated with 10 μM tirilazad and 75 μg/ml Bothrops alternatus venom did not show separation of the basement membrane. Moderate presence of altered epidermal cells was observed, predominantly moderate nuclear pyknosis.

Tissue incubated with 20 μM tirilazad showed intense separation of the basement membrane, while epidermal cells were normal and showed preserved cytoplasmic and nuclear structure.

Tissue incubated with 20 μM tirilazad and 75 μg/ml Bothrops alternatus venom showed intense separation of the basement membrane and intense presence of altered epidermal cells, predominantly intense nuclear pyknosis.

These results demonstrate that tirilazad provides a protective and/or therapeutic effect on cellular structure in hoof tissue.

TABLE 4
Results of Tirilazad Administration

| | Separation of | Cell | Pyknosis or |
|---|---|---|---|
| | Basement Membrane | Nuclei | Karyopyknosis* |
| Control | No | Present - | No |
| | | Normal | |
| Bothrops venom | +++ | Present - | +++ |
| (75 μg/ml) | | Abnormal | |
| | | +++ | |
| Tirilazad | No | Present - | No |
| (1 μM) | | Normal | |
| Venom (75 μg/ml) + | No | Present - | +++ |
| Tirilazad (1 μM) | | Abnormal | |
| | | +++ | |
| Tirilazad (3 μM) | No | Present - | + |
| | | Abnormal | |
| | | + | |
| Venom (75 μg/ml) + | + | Present - | +++ |
| Tirilazad (3 μM) | | Abnormal | |
| | | +++ | |
| Tirilazad (10 μM) | No | Present - | No |
| | | Normal | |
| Venom (75 μg/ml) + | No | Present - | ++ |
| Tirilazad (10 μM) | | Abnormal | |
| | | ++ | |
| Tirilazad (20 μM) | +++ | Present - | No |
| | | Normal | |
| Venom (75 μg/ml) + | +++ | Present - | +++ |
| Tirilazad (20 μM) | | Abnormal | |
| | | +++ | |

*Pyknosis or karyopyknosis is the irreversible condensation of chromatin in the nucleus of a cell undergoing necrosis or apoptosis; indicates cell death.

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

All references, patents or applications, U.S. or foreign, cited in the application are hereby incorporated by reference as if written herein in their entireties. Where any inconsistencies arise, material literally disclosed herein controls.

1. A method for treating or preventing a disorder associated with undesirable immune inflammatory activity leading to protease activity in a subject in need thereof, the method comprising administering to a subject a therapeutically or prophylactically effective amount of a compound of Formula I: 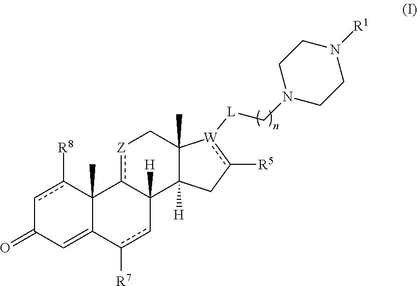
or a therapeutically acceptable salt thereof, wherein
W is chosen from C and CR⁴;
Z is CR⁶;
R¹ is heteroaryl, optionally substituted by one or more R² groups;
each R² is independently chosen from heterocycloalkyl and amino, either of which may be optionally substituted by one or more R³ groups;
each R³ is independently chosen from alkyl and allyl;
R⁴ is chosen from H, hydroxyl, and alkyl;
R⁵ is chosen from H and alkyl;
R⁶ is chosen from H, hydroxyl, and oxo;
R⁷ is chosen from H, halo, and alkyl;
R⁸ is chosen from H and hydroxyl;
L is chosen from —CH₂—, —C(O)—, and —C(CH₃)H—; and
n is chosen from 1, 2, or 3.

2. The method of claim 1, wherein R¹ is chosen from pyridinyl and pyrimidinyl, either of which may be optionally substituted by one or two R² groups.

3. The method of claim 2, wherein R¹ is pyrimidinyl, substituted by one or two R² groups.

4. The method of claim 3, wherein each R² is independently chosen from pyrrolidinyl, morpholinyl, diethylamino, amino, dimethylamino, piperazinyl, 4-methyl-1-piperazinyl, piperidinyl, allylamino, and ethylamino.

5. The method of claim 4, wherein each R² is pyrrolidinyl.

6. The method of claim 5, wherein R⁴ is H.

7. The method of claim 6, wherein R⁵ is H.

8. The method of claim 6, wherein R⁵ is methyl.

9. The method of claim 8, wherein R⁶, R⁷, and R⁸ are H, L is —C(O)—, and n is 1.

10. The method of claim 1, wherein the compound of Formula I is chosen from: 21-[4-[2-amino-6-(diethylamino)-4-pyrimidinyl]-1-piperazinyl]-17α-hydroxypregna-4,9(11)-diene-3,20-dione, 17α-hydroxy-21-[4-[2,6-bis(dimethylamino)-4-pyrimidinyl]-1-piperazinyl]pregna-4,9(11)-diene-3,20-dione, 21-[4-[2-(diethylamino)-6-(1-pyrrolidinyl)-4-pyrimidinyl]-1-piperazinyl]-17α-hydroxypregna-4,9(11)-diene-3,20-dione, 17α-hydroxy-21-[4-[2-(diethylamino)-6-(4-methyl-1-piperazinyl)(4-pyrimidinyl)]-1-piperazinyl]pregna-4,9(11)-diene-3,20-dione, 17α-hydroxy-21-[4-[2,6-bis(diethylamino)-4-pyrimidinyl]1-piperazinyl]pregna-4,9(11)-diene-3,20-dione, 1α-hydroxy-21-[4-[2-(diethylamino)-6-(1-piperidinyl)-4-pyrimidinyl]-1-piperazinyl]pregna-4,9(11)-diene-3,20-dione, 21-[4-[2,6-bis(diethylamino)4-pyrimidinyl]-1-piperazinyl]-17α-hydroxy-16α-methylpregna-1,4,9(11)-triene-3,20-dione, 17α-hydroxy-21-[4-[2,6-bis(4-methyl-1-piperazinyl)(4-pyrimidinyl)]-1-piperazinyl]pregna-4,9(11)-diene-3,20-dione, 17α-hydroxy-6α-methyl-21[4-[2,6-bis-(1-pyrrolidinyl)-4-pyrimidinyl]-1-piperazinyl]pregna-1,4,9(11)-triene-3,20-dione, 21-[4-[2,6-bis(diethylamino)-4-pyrimidinyl]-1-piperazinyl]-11α,17α-dihydroxypregn-4-ene-3,20-dione, 17α-hydroxy-21-[4-[2,6-bis(diethylamino)-4-pyrimidinyl]-1-piperazinyl]-pregn-4-ene-3,20-dione, 21-[4-[2,6-bis(diethylamino)-4-pyrimidinyl]-1-piperazinyl]-17α-hydroxy-6α-methylpregna-1,4,9(11)-triene-3,20-dione, 17α-hydroxy-21-[4-[2,6-bis(1-pyrrolidinyl)-4-pyrimidinyl]-1-piperazinyl]pregna-4,9(11)-diene-3,20-dione, 21-[4-[2,6-bis(diethylamino)-4-pyrimidinyl]-1-piperazinyl]-11α-hydroxypregn-4-ene-3,20-dione, 21-[4-[2,6-bis(diethylamino)-4-pyrimidinyl]-1-piperazinyl]-11α,17.alpha.-dihydroxypregn-4-ene-3,20-dione, 17α-hydroxy-16α-methyl-21-[4-[2,6-bis-(1-pyrrolidinyl)-4-pyrimidinyl]-1-piperazinyl]pregna-1,4,9(11)-triene-3,20-dione, 17α-hydroxy-21-[4-[2,6-bis(1-pyrrolidinyl)-4-pyrimidinyl]-1-piperazinyl]pregna-1,4,9(11)-triene-3,20-dione, 21-[4-[2,6-bis(diethylamino)-4-pyrimidinyl]-1-piperazinyl]-17α-hydroxypregna-1,4,9(11)-triene-3,20-dione, 21-[4-[4,6-bis(diethylamino)-2-pyrimidinyl]-1-piperazinyl]-17α-hydroxypregna-1,4,9(11)-triene-3,20-dione, 21-[4-[2,6-bis(diethylamino)-4-pyrimidinyl]-1-piperazinyl]-16α-methylpregna-1,4,9(11)-triene-3,20-dione, 21-[4-[2,6-bis(diethylamino)-4-pyrimidinyl]-1-piperazinyl]-11α-hydroxy-16α-methylpregna-1,4-diene-3,20-dione, 21-[4-[2,6-bis(diethylamino)-4-pyrimidinyl]-1-piperazinyl]-16α-methylpregna-1,4-diene,3,20-dione, 16α-methyl-21-[4-[2,6-bis(1-pyrrolidinyl)-4-pyrimidinyl]-1-piperazinyl]pregna-1,4,9(11)-triene-3,20-dione, 11α-hydroxy-16α-methyl-21-[4-[2,6-bis(1-pyrrolidinyl)-4-pyrimidinyl]piperazinyl]pregna-1,4-diene-3,20-dione, 16α-methyl-21-[4-[2,6-bis(1-pyrrolidinyl)-4-pyrimidinyl]-1-piperazinyl]pregna-1,4-diene-3,20-dione, 16α-methyl-21-[4-[2,6-bis(4-morpholino)-4-pyrimidinyl]-1-piperazinyl]pregna-1,4,9(11)-triene-3,20-dione, 11α-hydroxy-16α-methyl-21-[4-[2,6-bis(4-morpholino)-4-pyrimidinyl]-1-piperazinyl]pregna-1,4-diene-3,20-dione, 16α-methyl-21-[4-[2,6-bis(4-morpholino)-4-pyrimidinyl]-1-piperazinyl]pregna-1,4-diene-3,20-dione, 21-[4-[2,6-bis(allylamino)-4-pyrimidinyl]-1-piperazinyl]-16α-methylpregna-1,4,9(11)-triene-3,20-dione, 21-[4-[2,6-bis(allylamino)-4-pyrimidinyl]-1-piperazinyl]-11α-hydroxy-16α-methylpregna-1,4-diene-3,20-dione, 21-[4-[2,6-bis(allylamino)-4-pyrimidinyl]-1-piperazinyl]-16α-methylpregna-1,4-diene-3,20-dione, 21-[4-[2,6-bis(1-pyrrolidinyl)-4-pyrimidinyl]-1-piperazinyl]pregn-4-ene-3,11,20-trione, 21-[4-[2,6-bis (1-pyrrolidinyl)-4-pyrimidinyl]-1-piperazinyl]pregna-4,9(11)-diene-3,20-dione, 21-[4-[2,6-bis(1-pyrrolidinyl)-4-pyrimidinyl]-1-piperazinyl]pregna-1,4-diene-3,20-dione, 21-[4-(2,6-bis(1-pyrrolidinyl)-4-pyrimidinyl)-1-piperazinyl]pregna-4,9(11)-diene-3,20-dione, 21-[4-(2,6-bis(4-morpholino)-4-pyrimidinyl)-1-piperazinyl]-17α-hydroxypregna-4,9(11)-diene-3,20-dione, 21-[4-(2,6-bis(1-pyrrolidinyl)-4-pyrimidinyl)-1-piperazinyl]pregna-4-en-3-one, 21-[4-(2,6-bis(1-pyrrolidinyl)-4-pyrimidinyl)-1-piperazinyl]pregn-4-en-3-one, 16α-methyl-21-[4-[2,6-bis(1-pyrrolidinyl)-4-pyrimidinyl]-1-piperazinyl]pregna-1,4,9(11)-triene-3,20-dione, 21-[4-(2,6-bis(1-pyrrolidinyl)-4-pyrimidinyl)-1-piperazinyl]pregna-1,4,9(11)-triene-3,20-dione, 21-[4-(2,6-bis(1-pyrrolidinyl)-4-pyrimidinyl)-1-piperazinyl]-20-methylpregna-1,4-dien-3-one, 21-[4-(2,6-bis(1-pyrrolidinyl)-4-pyrimidinyl)-1-piperazinyl]pregna-1,4,9(11),16-tetraene-3,20-dione, 21-[4-[2,6-bis(4-morpholino)-4-pyrimidinyl]-1-piperazinyl]pregna-1,4-diene-3,20-dione, 21-[4-[2,6-bis(diethylamino)-4-pyrimidinyl]-1-piperazinyl]-6α-fluoro-17α-hydroxy-160-methylpregna-4,9(11)-diene-3,20-dione, 6α-fluoro-17α-hydroxy-160-methyl-21-[4-[2,6-bis(1-pyrrolidinyl)-4-pyrimidinyl]-1-piperazinyl]pregna-4,9(11)-diene-3,20-dione, 16α-methyl-21-[4-[2,6-bis(1-pyrrolidinyl)-4-pyrimidinyl]-1-piperazinyl]pregna-1,4-diene-3,20-dione, 21-[4-(2,6-bis(1-pyrrolidinyl)-4-pyrimidinyl)-1-piperazinyl]-16α,17α-dimethylpregna-1,4,9(11)-triene-3,20-dione, 16α-methyl-21-[4-[2,6-bis-(1-pyrrolidinyl)-4-pyrimidinyl]-1-piperazinyl]pregna-1,4,6,9(11)-tetraene-3,20-dione, 16α-methyl-170-(1-oxo-4-[4-[2,6-bis(1-pyrrolidinyl)-4-pyrimidinyl]-1-piperazinyl]butyl)-androsta-4,9(11)-dien-3-one, 21-[4-[5,6-bis(diethylamino)-2-pyridinyl]-1-piperazinyl]-16α-methylpregna-1,4,9(11)-triene-3,20-dione, 21-[4-[3-(ethylamino)-2-pyridinyl]piperazinyl]-16α-methylpregn-1,4,9(11)-triene-3,20-dione, 21′-[4-(2,6-bis(1-pyrrolidinyl)-4-pyrimidinyl)-1-piperazinyl]pregna-1,4,9(11),16-tetraene-3,20-dione, or a therapeutically acceptable salt thereof.

11. The method of claim 10, wherein the compound of Formula I is 16α-methyl-21-[4-[2,6-bis(1-pyrrolidinyl)-4-pyrimidinyl]-1-piperazinyl]pregna-1,4,9(11)-triene-3,20-dione, or a therapeutically acceptable salt thereof.

12. The method of claim 1, further comprising administration of an anti-hemorrhagic peptide.

13. The method of claim 1, further comprising administration of an anti-diabetic compound.

14. The method of claim 13, wherein the anti-diabetic drug is a sodium-glucose transport protein 2 (SGLT2).

15. The method of claim 14, wherein the SGLT2 inhibitor is selected from canagliflozin, canagliflozin/metformin, dapagliflozin, dapagliflozin/metformin, dapagliflozin/saxagliptin, dapagliflozin/saxagliptin/metformin, empagliflozin, empagliflozin/linagliptin, empagliflozin/linagliptin/metformin, empagliflozin/metformin, ertugliflozin, ertugliflozin/metformin, ertugliflozin/sitagliptin, Farxiga, Glyzambi, Invokamet, Invokamet-XR, Invokana, Jardiance, Qtern, Qternmet XR, Segluromet, Steglatro, Steglujan, Synjardy, Synjardy XR, Trijardy XR, and Xigduo XR.

16. The method of claim 1, wherein the subject is an ungulate.

17. The method of claim 16, wherein the ungulate is selected from equidae, bovinae, suidae, deer, ovis, and capra.

18. The method of claim 17, wherein the ungulate is a horse.

19. The method of claim 1, wherein the disorder is laminitis, equine chronic lung disease, equine osteoarthritis disease, equine septic joint disease, equine colic disease, equine chronic obstructive pulmonary disease, equine ulcerative colitis, equine Crohn's disease, equine inflammatory bowel disease.

20-27. (canceled)

28. The method of claim 1, wherein the compound of Formula I, or a therapeutically acceptable salt thereof, is administered at a dose of from about 0.1 μg to about 100 mg per kilogram body weight per day.

29. The method of claim 1, wherein the compound of Formula I, or a therapeutically acceptable salt thereof, is administered intravenously or by intramuscular depot delivery.

30. The method of claim 12, wherein the anti-hemorrhagic peptide is administered at a dose of from about 0.1 μg to about 100 mg per kilogram body weight of the subject per day.

31. The method of claim 30, wherein the anti-hemorrhagic peptide is administered at a dose of from about 1 μg to about 50 mg per kilogram body weight of the subject per day.

32. The method of claim 14, wherein the SGLT2 inhibitor is administered at a dose of from about 5 mg to about 300 mg.

33. A method for preventing or treating a condition associated with a gastrointestinal injury, disease, or ulcer in an animal in need thereof, comprising administering to the animal a therapeutically or prophylactically effective amount of a compound of Formula I: 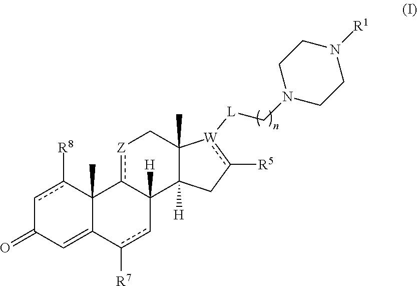
or a therapeutically acceptable salt thereof, wherein
W is chosen from C and CR⁴;
Z is CR⁶;
R¹ is heteroaryl, optionally substituted by one or more R² groups;
each R² is independently chosen from heterocycloalkyl and amino, either of which may be optionally substituted by one or more R³ groups;
each R³ is independently chosen from alkyl and allyl;
R⁴ is chosen from H, hydroxyl, and alkyl;
R⁵ is chosen from H and alkyl;
R⁶ is chosen from H, hydroxyl, and oxo;
R⁷ is chosen from H, halo, and alkyl;
R⁸ is chosen from H and hydroxyl;
L is chosen from —CH₂—, —C(O)—, and —C(CH₃)H—; and
n is chosen from 1, 2, or 3.

34. The method of claim 33, wherein the condition associated with a gastrointestinal injury, disease, or ulcer is a dental or oral wound; a peptic ulceration of the duodenum, stomach or esophagus; an inflammatory bowel disease; an ulcer associated with stress conditions; damage to the lining of the alimentary tract; inadequate gut function or damage to the gut associated with prematurity; a diarrheal condition; a food intolerance; a cancer of the gastrointestinal tract; surgically induced damage to the gut; damage due to esophageal reflux; a condition associated with loss of gut barrier function; a congenital condition resulting in inadequate gastrointestinal function or damage; or an autoimmune disease that affects the gut.
